(19) United States
(12) Patent Application Publication
(10) Pub. No.: US 2015/0190232 A1
(43) Pub. Date: Jul. 9, 2015

(54) ACETABULUM FOR A HIP PROSTHESIS

(71) Applicant: RV FINANCES, Rillieux-la-Pape (FR)

(72) Inventors: Richard Vandevelde, Rillieux-la-Pape (FR); Aurélien Bignon, Caluire Et Cuire (FR); Delphine Cordier, Lyon (FR)

(73) Assignee: RV FINANCES, Rillieux-la-Pape (FR)

(21) Appl. No.: 14/413,373

(22) PCT Filed: Jul. 11, 2013
(86) PCT No.: PCT/FR2013/051668

§ 371 Date: Jan. 7, 2015

(30) Foreign Application Priority Data
Jul. 11, 2012 (FR) 1256662

(51) Int. Cl. A61F 2/34; A61L 27/54; A61L 27/16; A61L 27/30; A61L 27/32

(52) U.S. Cl. CPC A61F 2/34; A61L 27/306; A61L 27/32; A61L 27/16; A61L 27/54; A61F 2310/00407; A61F 2310/00958; A61L 2300/428; A61L 2300/112; A61L 2300/102; A61L 2300/608; A61L 2420/02; A61L 2420/08

(57) ABSTRACT

The invention concerns an acetabulum for a hip prosthesis made from a biocompatible polymer, said acetabulum being covered with a coating which comprises a first layer of titanium applied to said biocompatible polymer and which further comprises, on the first layer, a second layer of titanium or of calcium phosphate, characterized in that:
the roughtness Rt of the first layer of titanium is between 40 and 500 μm,
said first layer of titanium has been applied by hot compression to said biocompatible polymer.
   Acetabula for hip prostheses.

### TECHNICAL FIELD

The present invention relates to an acetabulum for a hip prosthesis.

### PRIOR ART

Most of acetabula are consisted of a metal cup ensuring the interface with the bone and an insert ensuring the contact with the femoral head, wherein the cup and the insert can be or not cemented together. It is generally PMMA (Polymethylmethacrylate) cement. Such prostheses have an excellent survival rate of 10 years (about 95% of them). However, after this decade, the risk of loosening of the prosthesis exponentially increases.

Not much acetabula have a survival rate higher than 20 years, and that for several reasons, among which:
The stiffness of the cup may favour a wear at its interface with the insert.
The scraps resulting from the wear and the rubbings between the parts of the acetabulum are not without serious consequences for the patient's health, because they may generate an osteolysis and a loosening of the implant.
The metal cup being too stiff with respect to the bone, a micro-mobility may be generated at the interface, as well as a bone atrophy by derivation of the stresses.
The more the metal cup is thick, the less available room remains for the insert, and consequently, the more the thickness of the latter has to be reduced. This has for effect to increase the stresses in the insert, and hence to accelerate the wear thereof.

To overcome these drawbacks, so-called “non-cemented one-piece” acetablula (i.e. the coating of the cup is directly fixed to the insert) have been developed. They are made from a biocompatible polymer such as polyethylene (for example, UHMWPE “Ultra-High-Molecular-Weight Polyethylene”), on which has been directly deposited a coating of pure titanium or an alloy of titanium. Thanks to this innovation, the acetabula are more durables, because they solve the problems due to the stiffness of the metal cups detailed hereinabove. Indeed, the whole of the acetabulum keeps the stiffness of the polyethylene, that is close to that of the bone.

Moreover, making a one-piece acetabulum (polyethylene insert coated with titanium) allows to increase the thickness of the polyethylene, by comparison with an insert that is coupled with a metal cup. This offers the advantage to relieve the mechanical stresses that are imposed to the cup and to lengthen the acetabulum lifetime.

However, such “non-cemented one-piece” acetabula presently available on the market are not fully satisfactory as regards the risk of titanium particle graining-out during and after the installation of the acetabulum in the patient's body. More precisely, the risk of titanium particle graining-out may occur under the effect of mechanical stresses during the installation of the acetabulum or under the effect of rubbings due to the patient's movements. These particles might then migrate into the body, in particular in the rubbing parts of the implant, and generate a catastrophic wear of the implant.

### DESCRIPTION OF THE INVENTION

The present invention overcomes these drawbacks inherent to the non-cemented one-piece acetabula known to date by proposing new non-cemented one-piece acetabula, made from a biocompatible polymer and whose coating has been optimized to present a lessen risk of titanium particle graining-out during and after their installation in the patient's body with respect to the non-cemented one-piece acetabula presently available in the market. Moreover, according to certain embodiments of the invention, the acetabula according to the invention have also the non-negligible advantage to be more osteoconductive than the non-cemented one-piece acetabula presently known and available in the market.

The present invention has for first object an acetabulum for a hip prosthesis made from a biocompatible polymer, said acetabulum being covered with a coating that comprises a first layer of titanium that has been applied by hot compression on said biocompatible polymer, said coating being characterized in that:
the roughness Rt of the first layer of titanium is comprised between 40 and 500 μm, preferably between 100 and 500 μm, or between 100 and 200 μm,
said coating further comprises on the first layer:
   a second layer of titanium or of a calcium phosphate;
   optionally, on said second layer, when the second layer is a layer of titanium, a third layer of a calcium phosphate.

The invention has also for object a method for manufacturing said acetabulum. BEST WAY OF IMPLEMENTING THE INVENTION

Within the framework of the present invention, “layer of titanium” means:
either a layer essentially consisted of pure titanium,
or a layer essentially consisted of an alloy of titanium. Preferably, it is the TA6V alloy (an alloy whose composition, in weight percent, is: titanium 90%, aluminium 6% and vanadium 4%).

Advantageously, the titanium used to make the first layer of the acetabulum coating is in the form of a powder whose grain size is comprised between 20 and 500 μm.

Within the framework of the invention, “layer of a calcium phosphate” means a layer essentially consisted of at least one calcium phosphate, possibly coupled with a bioactive glass.

Preferably, the calcium phosphate is hydroxyapatite, brushite or a mixture of hydroxyapatite and brushite.

Within the framework of the present invention, “biocompatible polymer” means a polymer able to be implanted in the human body without being rejected by the patient or causing harmful reactions.

Moreover, the biocompatible polymers used within the framework of the invention have:
such a mechanical strength that the acetabulum ensures its function of prosthesis once implanted in the patient's body at the hip,
such a thermal resistance that said biocompatible polymer resists to the temperatures to which it is submitted during the application of the different layers of the coating.

Advantageously, the biocompatible polymer is chosen in the group consisted by the ultra-high-molecular-weight polyethylene (abbreviated “UHMWPE”), the polyarylether-ketone (abbreviated “PEAK”) such as the polyetheretherketones (abbreviated “PEEK”).

Preferably, the biocompatible polymer is the ultra-high-molecular-weight polyethylene (abbreviated “UHMWPE”).

Moreover, the UHMWPE may be non-cross-linked, cross-linked, possibly charged with antioxidants. The antioxidants are advantageously chosen among the carotenoids, the vitamins A, B, C, E (tocopherols) and K, the polyphenols, the BHT, the propyl, octyl and dodecyl gallates, the lipoic acid, the dihydroxylipoic acid, the amines, the hydroquinone, the coenzyme Q10 and the glutathione.

The UHMWPE is possibly charged with carbon. The carbon may have been mixed in the UHMWPE. Or the UHMWPE is in the form of a two-layer material, i.e. a first layer has been charged with carbon and the second layer is consisted of pure UHMWPE.

According to the embodiment of the invention, the biocompatible polymer is PEEK charged with carbon.

The roughness measurement allows to appreciate a state of surface. It quantifies the variations of height of a profile of the surface. Within the framework of the present invention, the roughness is a significant physical parameter because the clinging of the acetabulum to the bone depends on the roughness of the acetabulum. The roughness participates to the clinging to the bone. The implementation of a sufficient roughness Rt, comprised between 40 and 500 μm, for the first layer of titanium, further contributes to thermally isolate the biocompatible polymer from the heat liable to be released by the deposition of the second layer, for example when the latter is applied by thermal spraying (for example hot plasma). Within the framework of the present invention, it is meant by total roughness Rt the sum of the highest of the peak heights of the profile and of the greatest of the valley depths of the profile, within the length of evaluation, as defined by the standard ISO4287.

More precisely, to measure the roughness of the last layer of coating of the acetabulum, when it is a layer of pure titanium or of titanium alloy, the following steps are performed:
cutting the acetabula along their slice, then embedding them in a resin such as a methylmethacrylate resin (for example: the RESIN 605 marketed by the LAM PLAN Ltd company) so as to protect the coating during the following step of polishing,
performing a polishing of the slice to obtain a neat observable surface,
making a series of photographies with a microscope,
analysing the photographies by means of an image processing software to measure the roughness of the last layer of coating of the acetabulum.

To measure the roughness of a layer of coating of the acetabulum when it is a layer of calcium phosphate, a roughness meter is used. For that purpose, a sensing pin (element intended to touch the surface) is displaced in contact with the measured surface. The roughness meter records the variations of height along the displacement of the sensing pin so as to obtain a value of the roughness.

The application of the first layer of titanium of the acetabulum coating by hot compression may be performed according to a cycle, with the following physical parameters:
a pressurization between 20 and 100 bars in a compression chamber,
a rising of the temperature up to about 200° C.

The hot compression technique to make a first coating layer of titanium on an acetabulum made from a biocompatible polymer is perfectly within the skill of one of ordinary skill in the art.

The thickness of the first layer of the acetabulum coating is advantageously comprised between 50 and 800 μm, preferably between 150 and 500 μm, and more preferentially between 150 and 250 μm.

Advantageously, when the titanium is used to make the first layer of the acetabulum coating has a so-called “standard” grain size (i.e. comprised between about 90 and 125 μm), the roughness of the first layer of titanium obtained at the end of the hot compression is comprised between 80 μm and 160 μm. The rate of covering of the biocompatible polymer by the first layer of titanium is then advantageously comprised between 60 and 80%. The thickness of this first layer of the acetabulum coating is then comprised between 150 μm and 500 μm, and more preferentially between 150 and 250 μm.

When the titanium used to make the first layer of the acetabulum coating has a greater grain size, i.e. comprised between about 200 and 500 μm, the roughness of the first layer of titanium obtained at the end of the hot compression is comprised between 180 and 500 μm. The rate of covering of the biocompatible polymer by the first layer of titanium is then advantageously comprised between 70 and 90%. The thickness of this first layer of the acetabulum coating is then comprised between 300 and 800 μm.

The rate of covering of the biocompatible polymer by the first layer of the acetabulum coating depends both on the thickness of the first layer of titanium applied and on the grain size thereof. The rate of covering is advantageously of at least 60%. According to an embodiment of the invention, the rate of covering is comprised between 60 and 80%.

In a particularly advantageous manner, the rate of covering is almost of 100%.

The first layer of titanium is relatively easy to peel from the biocompatible polymer of the acetabulum, because each grain of titanium is anchored individually in the biocompatible polymer. The grain of titanium includes nibs. During the phase of hot compression, the biocompatible polymer melt around the grain of titanium by entering into its nibs. The grain of titanium is hence held by the mechanical force exerted by the biocompatible polymer at the contact thereof. Hence, for a grain of titanium to be loosened, this force should be sufficient to deform the biocompatible polymer in such a way to release it from the biocompatible polymer.

The particles of titanium anchored in the biocompatible polymer form a rough coating.

The first layer of the acetabulum coating has the following functions:
it ensures the clinging of the coating on the biocompatible polymer.
it serves as a support for the second layer of the coating of the acetabulum according to the invention.

Hence, the roughness of the first layer of the acetabulum coating is an essential characteristic of the present invention, because this first layer serves as a support for the second layer of the coating of the acetabulum according to the invention. If the first layer is not rough enough, the deposition of the second layer of the coating of the acetabulum according to the invention cannot be performed correctly.

The roughness of the second layer of the acetabulum coating is advantageously comprised between 30 and 400 μm, preferably between 100 and 400 μm, or preferentially between 100 and 180 μm.

The thickness of the second layer of the acetabulum coating may be comprised between 5 and 500 μm, preferably between 10 and 400 μm, or preferentially between 10 and 250 μm.

More precisely, the thickness of the second layer of the coating of the acetabulum according to the invention is advantageously comprised between 5 and 200 μm, when the second layer is a layer of calcium phosphate.

The thickness of the second layer of the coating of the acetabulum according to the invention is advantageously comprised between 50 and 500 μm, when the second layer is a layer of titanium.

According to an advantageous embodiment of the invention, when it is a second layer of titanium, the second layer is applied by plasma spraying, and for example hot plasma spraying.

When it is a second layer of calcium phosphate, the second layer of the acetabulum coating is advantageously applied by means of one of the following techniques:
plasma spraying,
electroplating,
soaking into a bath of calcium phosphate.

It is to be noted that, when the second layer of the acetabulum coating is made by plasma spraying, the thickness of the first layer has to be high enough to isolate the biocompatible polymer (that is temperature sensitive) from the material (titanium, titanium alloy such as TA6V or calcium phosphate) sprayed by plasma spraying. Indeed, said material is molten at the time of deposition by plasma spraying. That is why it is necessary that it is not in direct contact with the biocompatible polymer so as not to degrade the latter. Hence, it is advisable that the thickness of the first layer of the acetabulum coating is thick enough to ensure a good isolation of the biocompatible polymer during a plasma spraying. The implementation of a roughness Rt comprised between 40 and 500 μm, and more preferentially between 100 and 500 μm, or between 100 and 200 μm contributes to this thermal isolation, by allowing to keep the molten material intended to form the second layer at a sufficient distance from the biocompatible material, which avoids to degrade the latter.

The soaking consists in dipping the acetabulum coated with the first layer of titanium into a bath of calcium phosphate, i.e. into a solution that contains a rate of calcium phosphate of the order of 40% in volume. The calcium phosphate contained in the solution then becomes fixed on the first layer of titanium, so as to constitute a second layer of the acetabulum coating.

As regards the electroplating technique, the calcium phosphate coating obtained by this technique has the advantages to be of constant thickness and to be present even in the porosities of the first layer of titanium.

The first layer of the acetabulum coating is advantageously consisted of a multitude of grains of titanium that are individually sealed in the biocompatible polymer. That is why the punctual stress required for loosening these grains of titanium is relatively not very high.

The second layer of the acetabulum coating is superimposed on the first layer.

On the other hand, the second layer is distributed in a continuous way (as the particles of pure titanium, of titanium alloy such as TA6V or of calcium phosphate are welded to each other) on the surface of the first layer. Whatever is one of the above-mentioned techniques of application, the second layer is formed upon contact with the grains of pure titanium or of titanium alloy such as TA6V of the first layer of the acetabulum coating by entering of the material of said second layer into the nibs of said grains of the first layer.

Due to the fact that the second layer is substantially continuous, the same punctual force applied at the surface of the coating generates different stresses, because they will be distributed within all the second layer. More precisely, the second layer being in contact with substantially all the grains of titanium of the first layer, the surface of contact that undergoes the stress is far greater. That is why a far greater force should be required to cause the loosening of the second layer of the acetabulum coating than to cause the loosening of the grains of titanium of the first layer, because the matter is, in the case of the second layer, to break metal bonds (if the material of the second layer is pure titanium and/or an alloy of titanium such as TA6V) or covalent bonds (if the material of the second layer is calcium phosphate).

The privileged contact of the material of the second layer with the grains of titanium of the first layer of the acetabulum coating prevents this second layer to cling off the acetabulum.

Hence, the second coating layer of the acetabulum according to the invention (made of pure titanium, and/or of an alloy of titanium such as TA6V, or of calcium phosphate) reinforces the first layer by generating a more robust coating of the acetabulum than if the coating were coated only with a first layer of titanium.

Moreover, when the second layer of the acetabulum coating is consisted of calcium phosphate, it further offers the advantage that the acetabulum according to the invention has a coating that ensures a stable support of growth for the bone.

All the interest of this second layer of the coating of the acetabulum according to the invention is then understood.

Optionally, the coating of the acetabulum according to the invention comprises a third layer of calcium phosphate when the second layer is a layer of titanium.

The thickness of this third layer is advantageously comprised between 5 and 200 μm.

The third layer is preferably applied on said second layer by:
plasma spraying,
electroplating,
soaking into a bath of calcium phosphate.

The third layer allows to obtain an osteconductive acetabulum.

In a particularly advantageous embodiment, the invention hence relates to an acetabulum coated with a first layer of titanium, itself covered with a second layer of titanium, itself covered with a third layer of calcium phosphate.

The invention has also for object a method for manufacturing an acetabulum such as described hereinabove. The manufacturing method comprises the following steps:
a) providing an acetabulum made from a biocompatible material, for example UHMPWE or PEAK;
b) coating said acetabulum with a first layer of titanium or of an alloy of titanium such as TA6V by hot compression;
c) coating said first layer of titanium with a second layer, said second layer being a layer of titanium or a layer of a calcium phosphate;
d) optionally, when the second layer is a layer of titanium, said second layer is covered with a third layer, said third layer being a layer of a calcium phosphate.

According to an embodiment of the manufacturing method, the second layer is a layer of a calcium phosphate and has been applied on the first layer by plasma spraying, electroplating or soaking into a bath of calcium phosphate.

According to another embodiment of the manufacturing method, the second layer is a layer of titanium and has been applied by plasma spraying on the first layer.

Hence, the invention implements, in a preferential alternative embodiment, a hot compression deposition of grains of titanium on the surface of an acetabulum made from a biocompatible polymer, so as to hence form at the surface of said acetabulum a first layer of titanium having a roughness comprised between 40 and 500 μm, and a thermal spraying (for example, by plasma) of a powder material (for example, a powder of titanium), such that said thus-sprayed powder forms a second layer on the first layer, while merging with the grains of titanium forming the first layer, and that without degrading the biocompatible polymer that is thermally protected, in particular by the roughness of the first layer, from the heat emitted by the molten powder sprayed on the first layer.

The invention hence allows to reduce the risk that the grains of titanium become detached from the biocompatible polymer, because said grains are advantageously bonded together by a second layer (of molten titanium for example), preferably substantially continuous, and without the need to increase the surface quantity of grains and without the biocompatible polymer has been thermally degraded.

### POSSIBILITY OF INDUSTRIAL APPLICATION

The invention finds its industrial application in the design, the manufacturing and the use of acetabula.

1. An acetabulum for a hip prosthesis made from a biocompatible polymer, said acetabulum being covered with a coating that comprises a first layer of titanium applied on said biocompatible polymer and that further comprises, on the first layer, a second layer of titanium or of a calcium phosphate, characterized in that:
a). the roughness Rt of the first layer of titanium is between 40 and 500 μm; and
b). said first layer of titanium has been applied by hot compression on said biocompatible polymer.

2. The acetabulum according to claim 1, characterized in that said roughness Rt of the first layer of titanium is between 100 and 500 μm.

3. The acetabulum according to claim 1, characterized in that said coating comprises on said second layer, when the second layer is a layer of titanium, a third layer of a calcium phosphate.

4. The acetabulum according to claim 1, characterized in that the biocompatible polymer is selected from the group consisting of the ultra-high-molecular-weight polyethylene (UHMWPE), non-cross-linked, cross-linked and/or charged with antioxidants preferably chosen among the carotenoids, the vitamins A, B, C, E (tocopherols) and K, the polyphenols, the BHT, the propyl, octyl and dodecyl gallates, the lipoic acid, the dihydroxylipoic acid, the amines, the hydroquinone, the coenzyme Q10 and the glutathione, the polyaryletherketone (PEAK) such as the polyetheretherketones (PEEK), charged or not with carbon.

5. The acetabulum according to claim 1, characterized in that the thickness of the first layer of titanium is between 50 and 800 μm.

6. The acetabulum according to claim 5, characterized in that the thickness of the first layer of titanium is between 150 and 500 μm.

7. The acetabulum according to claim 1, characterized in that the rate of covering of the first layer of titanium on the biocompatible polymer is at least 60%.

8. The acetabulum according to claim 1, characterized in that the roughness of the second layer is between 30 and 400 μm.

9. The acetabulum according to claim 8, characterized in that the roughness of the second layer is between 100 and 400 μm.

10. The acetabulum according to claim 1, characterized in that the thickness of the second layer of the acetabulum coating is between 5 and 500 μm.

11. The acetabulum according to claim 10, characterized in that the thickness of the second layer of the acetabulum coating is between 10 and 400 μm.

12. The acetabulum according to claim 1, characterized in that the coating of said acetabulum comprises a second layer of titanium and a third layer of a calcium phosphate whose thickness is between 5 and 200 μm.

13. The acetabulum according claim 1, characterized in that said first layer consists of a multitude of grains of titanium that are individually sealed in the biocompatible polymer.

14. The acetabulum according to claim 13, characterized in that said second layer is substantially continuous and is in contact with substantially all the grains of titanium of the first layer.

15. A method for manufacturing an acetabulum according to claim 1, comprising the following steps:
a) providing an acetabulum made from a biocompatible material, for example UHMPWE or PEAK;
b) coating said acetabulum with a first layer of titanium;
c) coating said first layer of titanium with a second layer, said second layer being a layer of titanium or a layer of a calcium phosphate;
said method being characterized in that said acetabulum is coated with said first layer of titanium by hot compression, the roughness Rt of the first layer of titanium being between 40 and 500 μm.

16. The method for manufacturing an acetabulum according to claim 15, characterized in that it comprises a step d) in which, when the second layer is a layer of titanium, said second layer is coated with a third layer, said third layer being a layer of a calcium phosphate.

17. The method for manufacturing an acetabulum according to claim 16, characterized in that the third layer has been applied on the second layer by plasma spraying, electroplating, or soaking into a bath of calcium phosphate.

18. The method for manufacturing an acetabulum according to claim 15, characterized in that the second layer is a layer of a calcium phosphate and has been applied on the first layer by plasma spraying, electroplating, or soaking into a bath of calcium phosphate.

19. The method for manufacturing an acetabulum according to claim 15, characterized in that the second layer is a layer of titanium and has been applied by plasma spraying on the first layer.

20. The method for manufacturing an acetabulum according to claim 15, characterized in that the step b) during which said acetabulum is coated with said first layer of titanium is performed by hot compression of a multitude of grains of titanium on said biocompatible polymer, so that said grains are individually sealed in the biocompatible polymer.
